(19) United States
(12) Patent Application Publication
(10) Pub. No.: US 2026/0364219 A1
(43) Pub. Date: Nov. 19, 2026

(54) STERILIZATION DEVICE FOR A FLUIDIC CONNECTOR FOR USE WITH A DIALYSIS APPARATUS

(71) Applicant: VIVANCE PTE LTD, Singapore (SG)

(72) Inventors: Suresha BELUR VENKATARAYA, Singapore (SG); Mandar Manohar GORI, Singapore (SG); Aaron Alexander AYU, Singapore (SG); Yau Luong KOH, Singapore (SG); Santosh VARGHESE, Singapore (SG); Peter Ferdinand HAYWOOD, Singapore (SG); Alessandro DONADIO, Singapore (SG); Ricardo AGUILAR GRAJEDA, Singapore (SG); Tze Chern Jason LIM, Singapore (SG)

(21) Appl. No.: 19/480,225

(22) PCT Filed: May 2, 2024
(86) PCT No.: PCT/SG2024/050287

§ 371 Date: Oct. 30, 2025

(30) Foreign Application Priority Data
May 3, 2023 (GB) 2306512.1

(51) Int. Cl. A61L 2/104; A61L 2/26; A61L 103/15

(52) U.S. Cl. CPC A61L 2/104; A61L 2/26; A61L 2103/15; A61L 2202/11; A61L 2202/122; A61L 2202/14

(57) ABSTRACT

The present disclosure generally relates to a sterilization device (200) for sterilizing a fluidic connector (300) for use with a dialysis apparatus. The sterilization device (200) comprises: a housing (210) for holding the fluidic connector (300); an adaptor (220) for connecting the fluidic connector (300) to enable fluid communication between the fluidic connector (300) and the dialysis apparatus while the adaptor (220) and fluidic connector (300) remain inside the housing (210); and broadband ultraviolet chip units (400) disposed in the housing (210 ), the broadband ultraviolet chip units (400) configured for emitting broadband ultraviolet radiation towards the adaptor (220) in the housing (210). The adaptor (220) is transmissive to the broadband ultraviolet radiation such that the broadband ultraviolet radiation enables sterilization of external and internal surfaces of the adaptor (220) and sterilization of the fluidic connector (300) that is connected into the adaptor (220).

### CROSS REFERENCE TO RELATED APPLICATION(S)

The present disclosure claims the benefit of United Kingdom Patent Application No. 2306512.1 filed on 3 May 2023, which is incorporated in its entirety by reference herein.

### TECHNICAL FIELD

The present disclosure generally relates to a sterilization device for a dialysis apparatus.

### BACKGROUND

Millions of people worldwide suffer from kidney-related problems, such as chronic kidney disease (CKD) and end-stage renal disease (ESRD), and they may require either dialysis, such as peritoneal dialysis or hemodialysis, or transplantation to maintain life. Hemodialysis is a procedure in which a dialysis machine takes over the function of the kidneys. In peritoneal dialysis, the peritoneum in the patient's abdomen acts as a natural filtration membrane. A common type of peritoneal dialysis is continuous ambulatory peritoneal dialysis (CAPD) wherein the patient uses patient transfer sets to attach to an APD cycler device through the patient line. Fresh dialysate is pumped into the patient's peritoneal cavity through the patient transfer set and an implanted catheter in the peritoneal cavity. The dialysate in the peritoneal cavity absorbs waste products from the patient's body and waste dialysate is discharged out from the peritoneal cavity.

Peritoneal dialysis offers greater flexibility for patients and allows them to perform dialysis at home. The patient transfer set has a fluidic connector 100 as shown in FIG. 1. The fluidic connector 100 is protected with a disposable cap when not in use and the fluidic connector 100 should be carefully handled by the patient to avoid contact with any surface. This is to prevent contamination of the fluidic connector 100 which has high exposure to the dialysate and can cause infection to the patient. However, there is still a small contamination risk as the fluidic connector 100 is prone to finger contacts by the patient handling it. To mitigate this risk, an iodine shield 110 is used to handle the fluidic connector 100, so that there is no direct contact between the patient's fingers and the fluidic connector 100, especially at the part where the fluidic connector 100 connects to the patient line. The iodine shield 110 contains a sponge 120 that is soaked with iodine to kill bacteria that may be present on the fluidic connector 100.

However, some areas of the fluidic connector 100, such as the internal bore, cannot be reached by the iodine and bacteria may remain in the internal bore where the dialysate flows through. Further, before the fluidic connector 100 is connected to the patient line, the iodine shield 110 must be removed. This leaves a window whereby the patient could accidentally mishandle the fluidic connector 100 when connecting it to the patient line, potentially causing contamination which can lead to patient infection.

Therefore, in order to address or alleviate at least one of the aforementioned problems and/or disadvantages, there is a need to provide an improved sterilization device for a dialysis apparatus.

### SUMMARY

According to a first aspect of the present disclosure, there is a sterilization device for sterilizing a fluidic connector for use with a dialysis apparatus. The sterilization device comprises: a housing for holding the fluidic connector within the housing, the fluidic connector connectable with a patient for dialysate flow with respect to the patient; an adaptor fixable within the housing, the adaptor configured for connecting the fluidic connector into the adaptor to enable fluid communication between the fluidic connector and the dialysis apparatus while the adaptor and fluidic connector remain inside the housing; and a set of broadband ultraviolet chip units disposed in the housing, the broadband ultraviolet chip units configured for emitting broadband ultraviolet radiation towards the adaptor in the housing. The adaptor is transmissive to the broadband ultraviolet radiation such that the broadband ultraviolet radiation enables sterilization of external and internal surfaces of the adaptor and sterilization of the fluidic connector that is connected into the adaptor.

According to a second aspect of the present disclosure, there is a sterilization device for sterilizing a fluidic connector for use with a dialysis apparatus. The sterilization device comprises: a housing for holding the fluidic connector within the housing, the fluidic connector connectable with a patient for dialysate flow with respect to the patient; the fluidic connector fixable within the housing, the fluidic connector configured for connecting with the dialysis apparatus to enable fluid communication between the fluidic connector and the dialysis apparatus while the fluidic connector remains inside the housing; and a set of broadband ultraviolet chip units disposed in the housing, the broadband ultraviolet chip units configured for emitting broadband ultraviolet radiation towards the fluidic connector in the housing. The fluidic connector is transmissive to the broadband ultraviolet radiation such that the broadband ultraviolet radiation enables sterilization of external and internal surfaces of the fluidic connector.

A sterilization device for a dialysis apparatus according to the present disclosure is thus disclosed herein. Various features, aspects, and advantages of the present disclosure will become more apparent from the following detailed description of the embodiments of the present disclosure, by way of non-limiting examples only, along with the accompanying drawings.

### BRIEF DESCRIPTION OF THE DRAWINGS

FIG. 1 is an illustration of sterilizing a fluidic connector using an iodine shield.

FIG. 2A and FIG. 2B are illustrations of a sterilization device according to one embodiment of the present disclosure.

FIG. 3A to FIG. 3C are illustrations of a sterilization device according to another embodiment of the present disclosure.

FIG. 4A and FIG. 4B are illustrations of connecting a fluidic connector to an adaptor of the sterilization device.

FIG. 5A to FIG. 5E are illustrations of broadband ultraviolet chip units of the sterilization device and broadband ultraviolet radiation emitted from the broadband ultraviolet chip units.

FIG. 6A to FIG. 6C illustrate results of studies performed on various bacteria species using the sterilization device.

FIG. 7A and FIG. 7B are illustrations of a wavelength spectrum from various ultraviolet sources.

FIG. 8A to FIG. 8D are illustrations of broadband ultraviolet radiation transmissivity through different materials.

FIG. 9 is an illustration of the adaptor of the sterilization device.

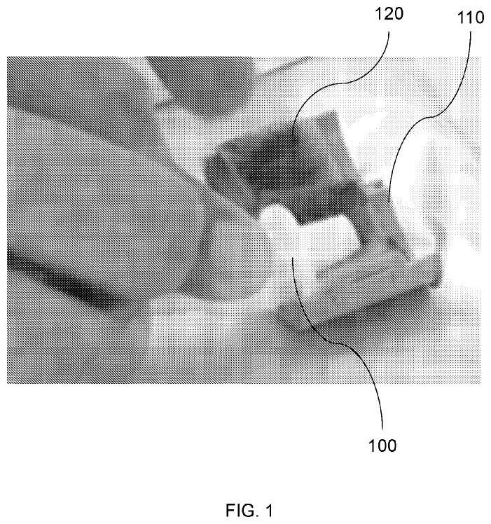

### DETAILED DESCRIPTION

For purposes of brevity and clarity, descriptions of embodiments of the present disclosure are directed to a sterilization device for a dialysis apparatus, in accordance with the drawings. While aspects of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognized by an individual having ordinary skill in the art, i.e. a skilled person, that the present disclosure may be practiced without specific details, and/or with multiple details arising from combinations of aspects of particular embodiments. In a number of instances, well-known systems, methods, procedures, and components have not been described in detail so as to not unnecessarily obscure aspects of the embodiments of the present disclosure.

In embodiments of the present disclosure, depiction of a given element or consideration or use of a particular element number in a particular figure or a reference thereto in corresponding descriptive material can encompass the same, an equivalent, or an analogous element or element number identified in another figure or descriptive material associated therewith.

References to “an embodiment/example”, “another embodiment/example”, “some embodiments/examples”, “some other embodiments/examples”, and so on, indicate that the embodiment(s)/example(s) so described may include a particular feature, structure, characteristic, property, element, or limitation, but that not every embodiment/example necessarily includes that particular feature, structure, characteristic, property, element or limitation. Furthermore, repeated use of the phrase “in an embodiment/example” or “in another embodiment/example” does not necessarily refer to the same embodiment/example.

The terms “comprising”, “including”, “having”, and the like do not exclude the presence of other features/elements/steps than those listed in an embodiment. Recitation of certain features/elements/steps in mutually different embodiments does not indicate that a combination of these features/elements/steps cannot be used in an embodiment.

As used herein, the terms “a” and “an” are defined as one or more than one. The use of “/” in a figure or associated text is understood to mean “and/or” unless otherwise indicated. The term “set” is defined as a non-empty finite organisation of elements that mathematically exhibits a cardinality of at least one (e.g. a set as defined herein can correspond to a unit, singlet, or single-element set, or a multiple-element set), in accordance with known mathematical definitions.

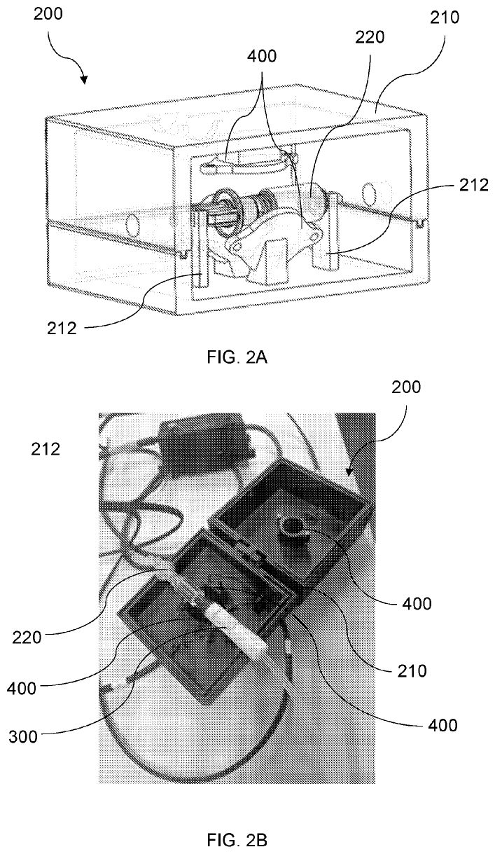

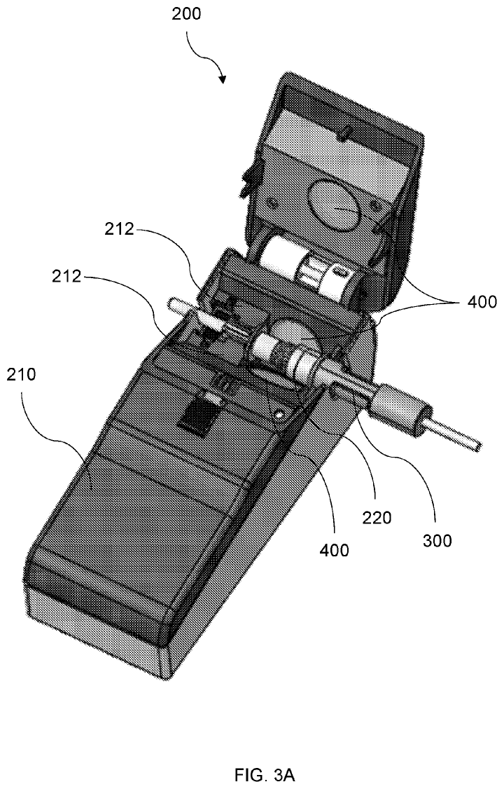

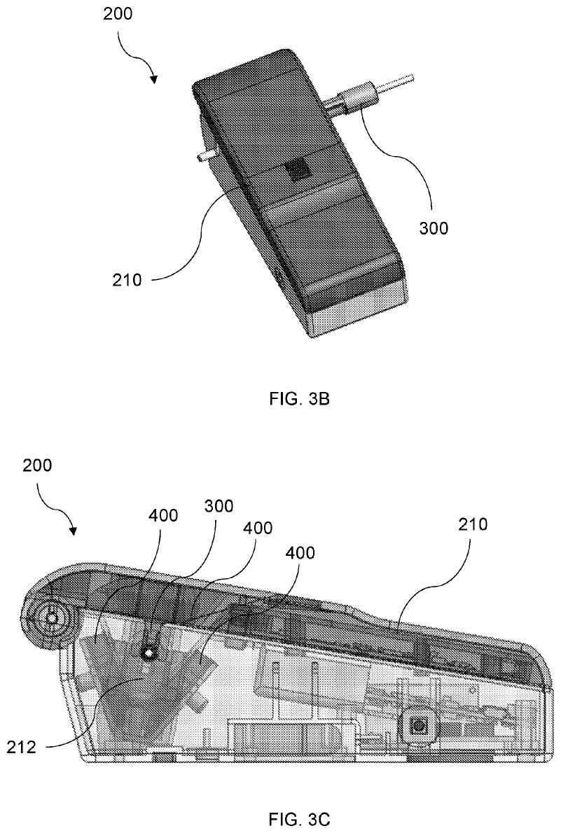

Representative or exemplary embodiments of the present disclosure describe a sterilization device 200 for sterilizing a fluidic connector 300 for use with a dialysis apparatus. The dialysis apparatus may be an apparatus for peritoneal dialysis or an apparatus for hemodialysis. FIG. 2A and FIG. 2B show one embodiment of the sterilization device 200, and FIG. 3A to FIG. 3C show another embodiment of the sterilization device 200.

The sterilization device 200 includes a housing 210 for holding the fluidic connector 300 within the housing 210, and includes an adaptor 220 fixable within the housing 210. For example, the housing 210 includes a supporting member 212 within the housing 210 and the adaptor 220 can be coupled to the supporting member 212 to fix the adaptor 220 in the housing 210. The adaptor 220 is configured for connecting the fluidic connector 300 into the adaptor 220 to enable fluid communication between the fluidic connector 300 and the dialysis apparatus while the adaptor 220 and fluidic connector 300 remain inside the housing 210. For example, the dialysis apparatus is a peritoneal dialysis apparatus and the fluidic connector 300 is part of the patient transfer set connected to patient's implanted catheter and the fluidic connector 300 is connectable via the adaptor 220 to the patient line of the peritoneal dialysis apparatus for dialysate flow with respect to the patient. The fluidic connection through the adaptor 220 enables dialysate to flow between the patient transfer set and the patient line.

The sterilization device 200 further includes a set of broadband ultraviolet chip units 400 disposed in the housing 210. The broadband ultraviolet chip units 400 are configured for emitting broadband ultraviolet radiation towards the adaptor 220 in the housing 210. More specifically, the adaptor 220 is transmissive to the broadband ultraviolet radiation such that the broadband ultraviolet radiation enables sterilization external and internal surfaces of the adaptor 220 as well as sterilization of the fluidic connector 300 that is connected into the adaptor 220. For example, the broadband ultraviolet radiation destroys most of the bacteria and endotoxins that are present on the fluidic connector 300.

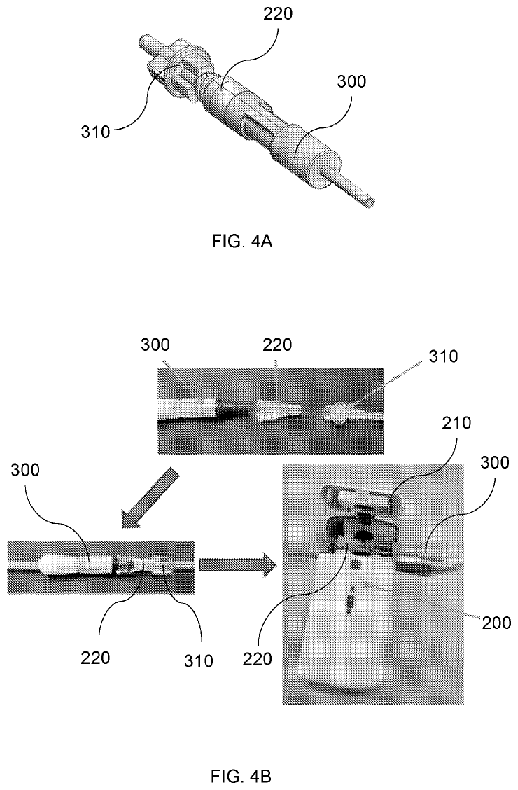

Before using the sterilization device 200, the patient connects the fluidic connector 300 of the patient transfer set into the adaptor 220. For example, the adaptor 220 has an internal orifice for receiving the fluidic connector 300, as shown in FIG. 4A and FIG. 4B. The patient can skip the iodine shield 110 and directly connect the fluidic connector 300 into the adaptor 220. The patient then places the adaptor 220 together with the connected fluidic connector 300 inside the housing 210, such as by coupling the adaptor 220 to the supporting member 212 for the broadband ultraviolet chip units 400 to sterilize the fluidic connector 300. The other end of the adaptor 220 is connected to the patient line 310 of the peritoneal dialysis apparatus.

The UV-transmissive adaptor 220 allows the broadband ultraviolet radiation to pass through it and target sterilization of the external and internal surfaces of the adaptor 220 where dialysate would contact during peritoneal dialysis. The broadband ultraviolet radiation also sterilizes less accessible areas of the fluidic connector 300, such as the fastening threads and internal bore, where the conventional method of using iodine cannot reach. The broadband ultraviolet radiation is thus able to sterilize all external and internal areas of the adaptor 220 and the fluidic connector 300.

In some embodiments, the sterilization device 200 includes a set of reflector units arranged in the housing 210 to uniformly distribute the broadband ultraviolet radiation around the adaptor 220. For example, the broadband ultraviolet chip units 400 may be positioned on one side of the adaptor 220, but the reflector units in the housing 210 enable the broadband ultraviolet radiation emitted from the broadband ultraviolet chip units 400 to be uniformly distributed around the adaptor 220, thereby allowing the adaptor 220 to receive equal intensity of the broadband ultraviolet radiation all around. The reflector units may be made of an ultraviolet-reflective material such as expanded polytetrafluoroethylene (ePTFE) or aluminum.

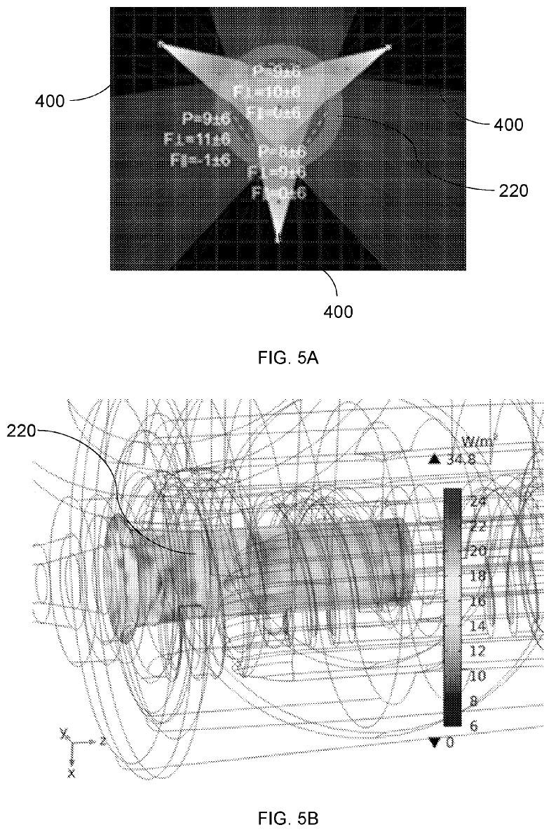

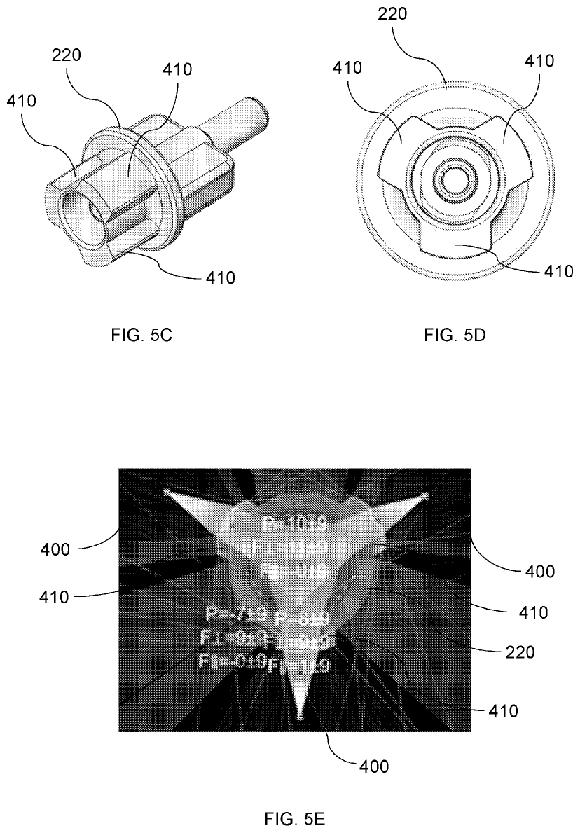

In some embodiments as shown in FIG. 2A and FIG. 3C the broadband ultraviolet chip units 400 are arranged in the housing 210 such that the broadband ultraviolet chip units 400 are equally distributed around the adaptor 220. For example and further as shown in FIG. 5A, the sterilization device 200 includes three broadband ultraviolet chip units 400 that are equally distributed around the adaptor 220, i.e. at 120° separations in between. This arrangement of the broadband ultraviolet chip units 400 allows for even distribution of the broadband ultraviolet radiation so that all areas of the fluidic connector 300 can be effectively sterilized. FIG. 5B shows a simulation of the broadband ultraviolet radiation received by the adaptor 220. All areas of the adaptor 220 can receive the broadband ultraviolet radiation, with an average intensity of 1.57 mW/cm², and no area has an intensity below 3.5 mW/cm². This shows that the broadband ultraviolet radiation can effectively reach and sterilize the fluidic connector 300 that is connected into the adaptor 220.

In some embodiments as shown in FIG. 5C to FIG. 5E, the sterilization device 200 includes a set of lenses 410 corresponding to the broadband ultraviolet chip units 400. For example, the lenses 410 are attached to the broadband ultraviolet chip units 400. For example, the lenses 410 are disposed around the adaptor 220. Each lens 410 is configured for focusing the broadband ultraviolet radiation from the respective broadband ultraviolet chip unit 400 into the adaptor 220. More specifically, the lenses 410 allow beam focusing for higher intensity broadband ultraviolet radiation into the adaptor 220, thereby improving the bacteria kill rate. For example, the adaptor 220 includes three lenses 410 that correspond to the three broadband ultraviolet chip units 400 equally distributed around the adaptor 220. Notably, the lenses 410 are also at 120° separations from each other. The supporting member 212 of the housing 210 is preferably configured to support the adaptor 220 in a fixed orientation that aligns the lenses 410 to the broadband ultraviolet chip units 400.

In some embodiments, the sterilization device 200 further includes a controller configured for controlling an intensity and/or a duration of the broadband ultraviolet radiation. For example, the controller controls the maximum intensity of the broadband ultraviolet radiation emitted from the broadband ultraviolet chip units 400. For example, the controller controls the duration when the adaptor 220 and fluidic connector 300 are exposed to the broadband ultraviolet radiation, such as 90 seconds or 2 minutes. The exposure duration may depend on the level of contamination of the fluidic connector 300, such as based on bacteria/endotoxins estimates.

The sterilization device 200 may include a sensor configured for measuring the intensity of the broadband ultraviolet radiation. The controller may be configured for receiving feedback from the sensor and adjusting the intensity and/or duration based on the feedback. For example, if the sensor detects that the broadband ultraviolet radiation is low, which if unchanged would result in poorer efficiency in sterilizing the fluidic connector 300, the controller may increase the intensity and/or extend the duration of the broadband ultraviolet radiation.

The controller may be configured for iteratively adjusting the intensity and/or duration based on a microbial log reduction model. The microbial log reduction model receives an initial input of a microbial estimate which estimates the bacteria count on the fluidic connector 300. For example, the bacteria count can be estimated using bacteria growth strips dipped against the surface of interest of the fluidic connector 300. The bacteria growth strips would change colour upon reacting with bacteria on the surface of interest, and the colour change can be compared against a colour chart to estimate the bacteria count.

The microbial log reduction model is able to achieve at least a 5-log reduction. This means that the bacteria can be reduced by at least 99.999%. Preferably, the microbial log reduction model is able to achieve a 6-log reduction. In this case, the microbial log reduction model calculates the minimum duration to kill at least 99.9999% of the bacteria, i.e. at least a 6-log kill rate which leaves behind one cell in one million, based on the initial microbial estimate and the initial intensity of the broadband ultraviolet radiation. The intensity is continuously measured by the sensor and fed back to the microbial log reduction model to iteratively adjust the exposure duration. The exposure duration is the time needed to achieve the 6-log kill rate. For example, an intensity of 35 mJ/cm² would require at least 22 seconds to achieve the 6-log kill rate. For example, an intensity of 46.7 mJ/cm² would require at least 30 seconds to achieve an 8-log kill rate.

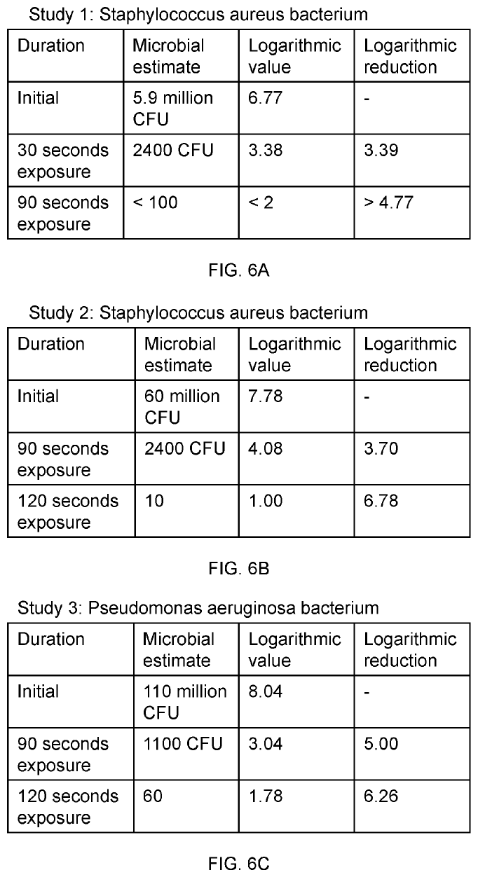

Some studies were performed on the microbial log reduction model to compare the exposure duration and the bacteria kill rate for various species of bacteria. As shown in FIG. 6A, Study 1 was performed to determine the kill rates of staphylococcus aureus bacterium at exposure durations of 30 seconds and 90 seconds. It was found that the 90-seconds exposure achieved less than the desired 6-log kill rate. As shown in FIG. 6B, Study 2 was performed to determine the kill rates of staphylococcus aureus bacterium at exposure durations of 90 seconds and 120 seconds. It was found that the 120-seconds exposure achieved more than the desired 6-log kill rate. As shown in FIG. 6C, Study 3 was performed to determine the kill rates of pseudomonas aeruginosa bacterium at exposure durations of 90 seconds and 120 seconds. It was found that the 120-seconds exposure achieved more than the desired 6-log kill rate. As described above, the broadband ultraviolet chip units 400 are configured to emit broadband ultraviolet radiation. Preferably, the broadband ultraviolet radiation is in the wavelength range of 230 to 340 nm. The broad spectrum of the ultraviolet radiation targets a wider range of bacteria and improves the bacteria kill rate. The broadband ultraviolet chip units 400 may include cathodoluminescent (CL) chips such as CL-based deep ultraviolet (DUV) chips. The broadband ultraviolet chip units 400 may be selected with specific peak wavelengths to optimize sterilization of the fluidic connector 300.

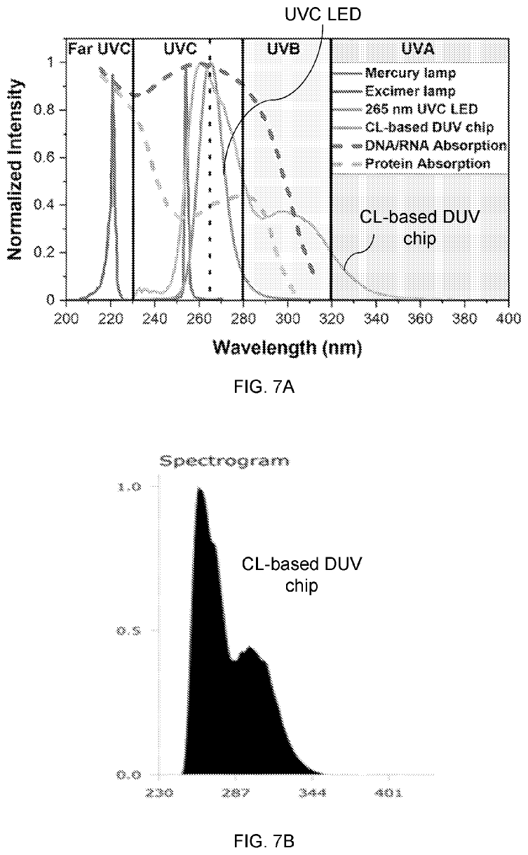

FIG. 7A and FIG. 7B show the wavelength range of different ultraviolet sources including the broadband ultraviolet chip units 400, i.e. CL-based DUV chips, as well as ultraviolet light emitting diodes (LEDs). Notably, the CL-based DUV chip has a wider wavelength range of about 230 nm to 340 nm with a peak wavelength at about 260 nm, whereas the UVC LED has a wavelength range of about 250 nm to 290 nm with a peak wavelength at about 265 nm. The wavelength profile of the UVC LED is also much sharper than the wavelength profile of the CL-based DUV chip. This means that the UVC LED is only efficient in emitting ultraviolet radiation in a narrow range of wavelengths, whereas the CL-based DUV chip is efficient in a wider range of wavelengths. Notably, at least 50% of the broadband ultraviolet radiation from the CL-based DUV chip is in the wavelength range of about 250 nm to 280 nm, whereas for the UVC LED, the 50% threshold corresponds to a narrower wavelength range of about 260 nm to 270 nm.

Various UV-transmissive materials may be used for the adaptor 220. For example, the adaptor 220 is made of fluorinated ethylene propylene (FEP). FEP is highly transmissive and highly stable to ultraviolet radiation and degrades minimally from exposure to the ultraviolet radiation. Other suitable materials that are highly transmissive and highly stable to ultraviolet radiation include, but are not limited to, cyclic olefin copolymers (COC), cyclic block copolymers (CBC), and polymethyl methacrylate (PMMA).

As the broadband ultraviolet radiation from the broadband ultraviolet chip units 400 has a wide spectrum range, the adaptor 220 should be made of a UV-transmissive material that allows substantially all of the broadband ultraviolet radiation to pass through. Preferably, the adaptor 220 is made of cyclic block copolymers (CBC). CBC is a class of thermoplastics with excellent optical transparency and chemical resistance, making them highly transmissive and highly stable to broadband ultraviolet radiation.

CBC may be formed from any suitable block copolymers. For example, CBC may be formed from styrene-conjugated dienes copolymers. Examples of suitable CBC include, but are not limited to, ViviOn™ (CBC) from USI Corporation. ViviOn™ (CBC) is a family of CBC formed from styrene-butadiene copolymers which are formed from poly(cyclohexylethylene) (PCHE) and ethylene-co-1-butene (EB). Without wishing to be bound by theory, the properties of ViviOn™ may be modified by adjusting the ratio of PCHE and EB.

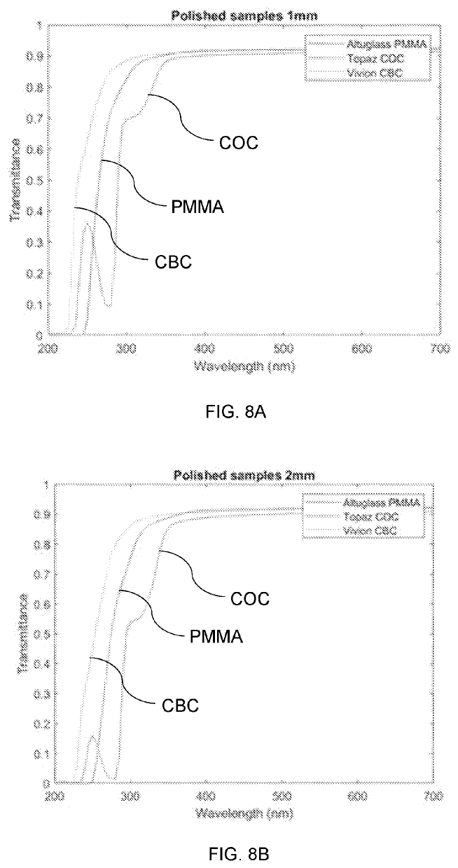

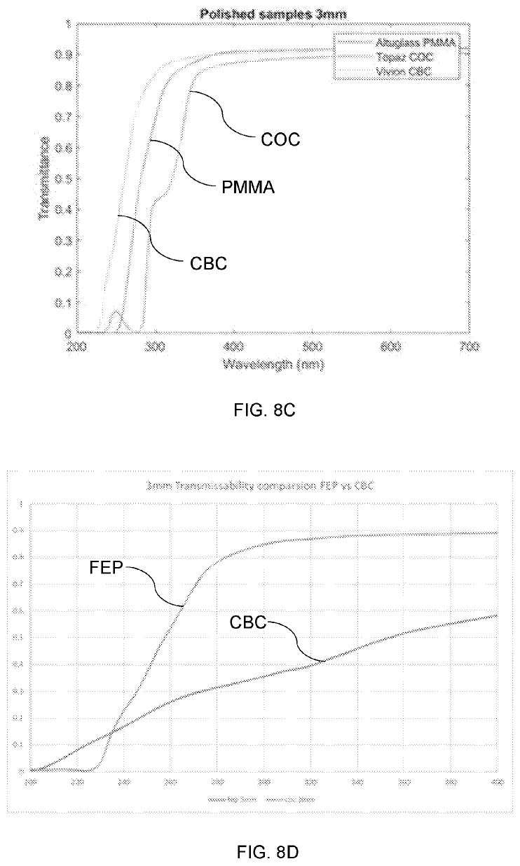

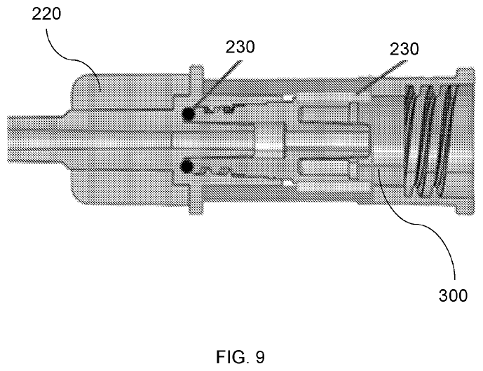

Different samples of FEP, PMMA, COC, and CBC materials having thicknesses of 1 mm, 2 mm, and 3 mm were tested for their ultraviolet transmittance properties. The results are shown in FIG. 8A to FIG. 8D. It was found that the CBC material is more transmissive to broadband ultraviolet radiation over a wider wavelength range. Referring to the 3 mm samples in FIG. 8C for example, at the 240 nm wavelength of the broadband ultraviolet radiation, 18% of the ultraviolet radiation passes through the FEP material, 0% of the ultraviolet radiation passes through the PMMA material, less than 0.1% of the ultraviolet radiation passes through the COC material, and 20% of the ultraviolet radiation passes through the CBC material. At the 250 nm wavelength of the broadband ultraviolet radiation, 20% of the ultraviolet radiation passes through the FEP material, 0% of the ultraviolet radiation passes through the PMMA material, less than 0.1% of the ultraviolet radiation passes through the COC material, and 30% of the ultraviolet radiation passes through the CBC material. Hence, the CBC material is superior over the FEP, PMMA, and COC materials in terms of transmissivity of broadband ultraviolet radiation.

However, ViviOn™ (CBC) is a brittle material and may crack easily when subjected to forces. For example, the adaptor 220 may crack or break if the patient uses excessive force in connecting the fluidic connector 300 into the adaptor 220. In order to attenuate this force, the adaptor 220 may include a resilient material 230 at its internal orifice, such as the internal threading that fastens to the fluidic connector 300. The resilient material 230 may include soft polymers, such as medical grade SEBS (styrene-ethylene-butylene-styrene) or TPE (thermoplastic elastomers). In addition to reducing stress build-up in the adaptor 220 which can cause the ViviOn™ (CBC) material to crack or break, the resilient material 230 also improves the fluidic seal between the fluidic connector 300 and the adaptor 220, thereby mitigating risk of fluid leakage.

Therefore, as described in various embodiments herein, the sterilization device 200 provides a way for the patient to self-sterilize the fluidic connector 300 of the patient transfer set. Unlike the conventional way of using iodine, the fluidic connector 300 can be sterilized after connecting the fluidic connector 300 into the adaptor 220 and before beginning peritoneal dialysis therapy. The sterilization device 200 lowers the contamination risk of the fluidic connector 300 and infection risk to the patient. With lower infection risk, the patient is less likely to suffer from medical conditions such as bacterial inflammation of the peritoneum and peritonitis disease.

Various embodiments herein describe the sterilization device 200 having the adaptor 220 configured for connecting with the fluidic connector 300 for use with the dialysis apparatus. In other embodiments, the sterilization device 200 does not have the adaptor 220. Instead, the sterilization device 200 includes the housing 210, the fluidic connector 300, and the broadband ultraviolet chip units 400. The fluidic connector 300 is directly connectable to the dialysis apparatus without the adaptor 220. For example, the dialysis apparatus is a peritoneal dialysis apparatus and the fluidic connector 300, which is part of the patient transfer set connected to patient's implanted catheter, is directly connectable to the patient line 310 of the peritoneal dialysis apparatus. The fluidic connector 300 enables dialysate to flow between the patient transfer set and the patient line 310.

More specifically, the fluidic connector 300 is fixable within the housing 210 and is configured for connecting with the dialysis apparatus to enable fluid communication between the fluidic connector 300 and the dialysis apparatus while the fluidic connector 300 remains inside the housing 210. The broadband ultraviolet chip units 400 are disposed in the housing 210 and configured for emitting broadband ultraviolet radiation towards the fluidic connector 300 in the housing 210. The fluidic connector 300 is transmissive to the broadband ultraviolet radiation such that the broadband ultraviolet radiation enables sterilization of external and internal surfaces of the fluidic connector 300.

The fluidic connector 300 in these embodiments is UV-transmissive like the adaptor 220 described further above. It will be appreciated that various aspects of the adaptor 220 described herein will apply similarly or analogously to the fluidic connector 300 in these embodiments.

Although various embodiments herein describe the sterilization device 200 for use with a peritoneal dialysis apparatus, it will be appreciated that the sterilization device 200 can be used for other dialysis apparatuses such as a hemodialysis apparatus. Various aspects of the sterilization device 200 for the peritoneal dialysis apparatus will apply similarly or analogously for the hemodialysis apparatus.

In the foregoing detailed description, embodiments of the present disclosure in relation to a sterilization device for a dialysis apparatus are described with reference to the provided figures. The description of the various embodiments herein is not intended to call out or be limited only to specific or particular representations of the present disclosure, but merely to illustrate non-limiting examples of the present disclosure. The present disclosure serves to address at least one of the mentioned problems and issues associated with the prior art. Although only some embodiments of the present disclosure are disclosed herein, it will be apparent to a person having ordinary skill in the art in view of this disclosure that a variety of changes and/or modifications can be made to the disclosed embodiments without departing from the scope of the present disclosure. Therefore, the scope of the disclosure as well as the scope of the following claims is not limited to embodiments described herein.

1. A sterilization device for sterilizing a fluidic connector for use with a dialysis apparatus, the sterilization device comprising:
a housing for holding the fluidic connector within the housing, the fluidic connector connectable with a patient for dialysate flow with respect to the patient;
an adaptor fixable within the housing, the adaptor configured for connecting the fluidic connector into the adaptor to enable fluid communication between the fluidic connector and the dialysis apparatus while the adaptor and fluidic connector remain inside the housing; and
a set of broadband ultraviolet chip units disposed in the housing, the broadband ultraviolet chip units configured for emitting broadband ultraviolet radiation towards the adaptor in the housing,
wherein the adaptor is made of cyclic block copolymers and is transmissive to the broadband ultraviolet radiation, such that the broadband ultraviolet radiation enables sterilization of external and internal surfaces of the adaptor and sterilization of the fluidic connector that is connected into the adaptor; and
wherein the adaptor comprises a resilient material to reduce stress forces in the adaptor and reduce fluid leakage from the adaptor.

2. The sterilization device according to claim 1, wherein the broadband ultraviolet chip units comprise cathodoluminescent chips.

3. The sterilization device according to claim 1, wherein the broadband ultraviolet radiation comprises a wavelength range of 230 to 340 nm.

4. The sterilization device according to claim 1, wherein at least 50% of the broadband ultraviolet radiation comprises a wavelength range of 250 nm to 280 nm.

5. The sterilization device according to claim 1, wherein the resilient material comprises a soft polymer.

6. The sterilization device according to claim 1, further comprising a set of reflector units arranged in the housing to uniformly distribute the ultraviolet radiation around the adaptor.

7. (canceled)

8. The sterilization device according to claim 1, further comprising a set of lenses corresponding to the ultraviolet units, each lens configured for focusing the ultraviolet radiation from the respective ultraviolet unit into the adaptor.

9. The sterilization device according to claim 1, further comprising a controller configured for controlling an intensity and/or a duration of the ultraviolet radiation.

10. The sterilization device according to claim 9, further comprising a sensor configured for measuring the intensity of the ultraviolet radiation, wherein the controller is further configured for receiving feedback from the sensor and adjusting the intensity and/or duration based on the feedback.

11. (canceled)

12. The sterilization device according to claim 9, wherein the controller is further configured for iteratively adjusting the intensity and/or duration based on a microbial log reduction model of at least 5-log reduction.

13. A sterilization device for sterilizing a fluidic connector for use with a dialysis apparatus, the sterilization device comprising:
a housing for holding the fluidic connector within the housing, the fluidic connector connectable with a patient for dialysate flow with respect to the patient;
the fluidic connector fixable within the housing, the fluidic connector configured for connecting with the dialysis apparatus to enable fluid communication between the fluidic connector and the dialysis apparatus while the fluidic connector remains inside the housing; and
a set of broadband ultraviolet chip units disposed in the housing, the broadband ultraviolet chip units configured for emitting broadband ultraviolet radiation towards the fluidic connector in the housing,
wherein the fluidic connector is made of cyclic block copolymers and is transmissive to the broadband ultraviolet radiation, such that the broadband ultraviolet radiation enables sterilization of external and internal surfaces of the fluidic connector; and
wherein the fluidic connector comprises a resilient material to reduce stress forces in the fluidic connector and reduce fluid leakage from the fluidic connector.

14. The sterilization device according to claim 13, wherein the broadband ultraviolet chip units comprise cathodoluminescent chips.

15. The sterilization device according to claim 13, wherein the broadband ultraviolet radiation comprises a wavelength range of 230 to nm.

16. The sterilization device according to claim 13, wherein at least 50% of the broadband ultraviolet radiation comprises a wavelength range of 250 nm to 280 nm.

17. The sterilization device according to claim 13, wherein the resilient material comprises a soft polymer.

18. The sterilization device according to claim 13, further comprising a set of reflector units arranged in the housing to uniformly distribute the ultraviolet radiation around the fluidic connector.

19. (canceled)

20. The sterilization device according to claim 13, further comprising a set of lenses corresponding to the ultraviolet units, each lens configured for focusing the ultraviolet radiation from the respective ultraviolet unit into the fluidic connector.

21. The sterilization device according to claim 13, further comprising a controller configured for controlling an intensity and/or a duration of the ultraviolet radiation.

22. The sterilization device according to claim 21, further comprising a sensor configured for measuring the intensity of the ultraviolet radiation, wherein the controller is further configured for receiving feedback from the sensor and adjusting the intensity and/or duration based on the feedback.

23. (canceled)

24. The sterilization device according to claim 21, wherein the controller is further configured for iteratively adjusting the intensity and/or duration based on a microbial log reduction model of at least 5-log reduction.
